(19) United States
(12) Patent Application Publication
(10) Pub. No.: US 2026/0021172 A1
(43) Pub. Date: Jan. 22, 2026

(54) Personalized Anticancer Vaccine Comprising Glycoengineered Tumour Cells or Tumour Cell Fragments

(71) Applicant: CARBOCALYX GMBH, Konstanz (DE)

(72) Inventors: Tamas Dolowschiak, Singen (DE); Györgyi Osztrovszky, Konstanz (DE); Ferenc Horvath, Konstanz (DE); Gyula Dekany, Sinnamon Park (AU)

(21) Appl. No.: 18/859,798

(22) PCT Filed: Apr. 27, 2023
(86) PCT No.: PCT/EP2023/061070

§ 371 Date: Oct. 24, 2024

(30) Foreign Application Priority Data
Apr. 28, 2022 (CH) CH000496/2022

(51) Int. Cl. A61K 39/00; A61K 39/385; A61P 35/00; C12N 5/00; C12N 5/09; G01N 33/574; G01N 33/68

(52) U.S. Cl. CPC A61K 39/001169; A61K 39/385; A61P 35/00; C12N 5/0006; C12N 5/0693; G01N 33/57492; G01N 33/6857; A61K 2039/6006; G01N 2400/00

(57) ABSTRACT

Disclosed herein is a glycoengineered tumor cell or a glycoengineered tumor cell fragment for use in treatment and/or prevention of cancer, in particular cancerous neoplasms, in a subject. The glycoengineered tumor cell or tumor cell fragment includes a tumor cell surface including one or more carbohydrate antigen moieties. Furthermore, a pharmaceutical composition including such a cell or cell fragment, a method for producing such a glycoengineered tumor cell or tumor cell fragment and a method of treatment of a subject including the administration of such a glycoengineered tumor cell or glycoengineered tumor cell fragment to a subject is disclosed.

### CROSS-REFERENCE TO RELATED APPLICATIONS

This application is the United States national phase of International Patent Application No. PCT/EP2023/061070 filed Apr. 27, 2023, and claims priority to Swiss Patent Application No. CH000496/2022 filed Apr. 28, 2022, the disclosures each of which are hereby incorporated by reference in their entireties.

### BACKGROUND OF THE INVENTION

### Field of the Invention

The present invention lies in the field of personalized cancer vaccination and relates in particular to a glycoengineered tumor cell or a glycoengineered tumor cell fragment for use in treatment and/or prevention of cancer, a pharmaceutical composition or use in treatment and/or prevention of cancer in a subject, a method for producing a glycoengineered tumor cell or tumor cell fragment and a method of treatment of a subject.

### Description of Related Art

Cancer is a leading cause of death worldwide. Current cancer treatments include chemotherapy, radiotherapy, surgery, palliative care, immunotherapy and alternative care. There are several types of cancer vaccines, which include nucleic-acid-based, peptide-based and cell-based vaccines. Current cancer vaccines make use of active immunotherapy, i.e., enhancing or inducing specific immune responses or repressing specific regulatory responses to eventually recognize, attack and destroy tumor cells. The identification of a growing number of tumor-associated antigens (TAAs) led to a broad collection of suitable targets for cancer immunotherapy and vaccine-based immunization. However, owing to the molecular heterogeneity in cancer, often less than 25% of treated individuals profit from the approved therapies. As an additional layer of heterogeneity, it is noteworthy to underline that not only individuals respond differently to particular vaccines and adjuvants but that the strength and length of the evoked immune responses and thereby the longevity of the desired protection significantly varies among individuals on a population scale.

Technological advances in cancer immunotherapy, molecular biology, and high-throughput analytical sciences such as genomics, proteomics and bioinformatics have paved the way to individualized medicine based on tailored treatment of patients as a solution for innovation in low efficacy-high cost drug development. Current personalized therapy approaches make particular use of genome sequencing (e.g., next generation sequencing (NGS)) to map genetic aberration in the entire genome of cancerous tissues, thus identifying their own unique set of mutations. Most commonly identified TAAs are selectively expressed on tumor cells but also may show some expression on non-tumor cells. Accordingly, TAAs are usually T-cell-independent and therefore only produce weak immune responses. For these reasons, focus has recently been put on the sequence-identification of tumor-specific neoantigens (i.e., neoepitopes), which are results of the accumulation of mutations in the tumor genome and are solely expressed on tumor cells, in contrast to TAAs (Sahin and Türeci 2018, doi 10.1126/science.aar7112).

The selection of an appropriate vaccine platform is a major factor for its success in a clinical setting. Several technological platforms are currently envisioned for the development of neoantigen-directed vaccines, including the utilization of bacterial (WO 2016/207859; WO 2016/191545) and viral vectors (US 2020/0138923 A1), nucleic acid vaccines, such as nanoparticles encapsulating mRNA-based vaccines and DNA plasmids (WO 2012/159754) synthetic peptides and antigen-loaded dendritic cells (Topalian et al. 2015, doi 10.1016/j.cell.2015.03.045). Nevertheless, the current personalized cancer vaccine approaches still face several limitations. Critical challenges for the clinical application of current personalized vaccines include identifying the most suitable clinical settings, reducing production turnaround time, upscaling manufacturing and ensuring affordability. Especially viral vectors and engineered attenuated bacterial vaccine formats are accompanied by complex manufacturing processes, but also by intricate immune responses against components of the microbial backbone. DNA plasmids face e.g., potential safety risks by insertional mutagenesis and mRNA-based vaccines face the risk of fast extracellular degradation if not protected by appropriate formulation.

Thus, there is a need to develop a personalized cancer vaccine and a method for producing a personalized cancer vaccine that meets the above challenges and is capable of inducing strong and long-lasting immune responses.

### SUMMARY OF THE INVENTION

It is the general object of the invention to advance the state of the art in the field of anticancer vaccination and in particular to overcome the disadvantages of the prior art fully or partly. In advantageous embodiments, a personalized anti-cancer vaccination is provided, which preferably shows increased safety, efficiency of manufacturing, high selectivity and/or avoids or at least diminishes the risk associated with other known techniques as described above.

The general object is achieved by the subject-matter and embodiments disclosed herein.

### BRIEF DESCRIPTION OF THE DRAWINGS

FIG. 1 is a graph showing comparative data of C57BL/6 mice treated with the indicated samples, as described below.

FIG. 2 is a graph showing in vitro data resembling the capacity of antigen presenting cells (APC) to boost T cell immunity in the absence or presence of immunogenic carbohydrates and their corresponding human natural anticarbohydrate antibodies, as described below.

FIG. 3 provides FACS plots of different malignant B16.F10 cells ligated with Melibiose (Mel) utilizing various linkages. The top row labelled with “A” shows the results of experiments conducted with murine antibodies for Mel detection and the bottom row labelled with “B” experiments conducted with human antibodies for Mel detection, as described below.

FIG. 4 provides graphs showing in vivo data for demonstrating the effect of a glycoengineered cell lysate comprising a glycoengineered tumor cell fragment as described herein on IgG and IgM levels in the subject, as described below.

FIG. 5 provides graphs showing in vivo data for demonstrating the effect of a glycoengineered cell lysate comprising a glycoengineered tumor cell fragment as described herein on IgG levels in the subject, as described below.

FIGS. 6a and 6b provide images showing the degree of lung metastasis in different test groups, as described below.

### DETAILED DESCRIPTION

A first aspect of the invention relates to a glycoengineered tumor cell or a glycoengineered tumor cell fragment for use in treatment and/or prevention of cancer, particularly cancerous neoplasms, in a subject. The glycoengineered, i.e. modified, tumor cell or tumor cell fragment comprises a tumor cell surface comprising one or more carbohydrate antigen moieties.

A glycoengineered tumor cell fragment as used herein may for example be a lysate or comprised in a lysate which has been obtained from a corresponding glycoengineered tumor cell. In some embodiments, the glycoengineered tumor cell fragment is administered as a lysate. Preferably, in embodiments in which a glycoengineered tumor cell fragment is employed, the glycoengineered tumor cell fragment is a separate tumor cell fragment and thus not a whole cell. For example, if the glycoengineered tumor cell fragment is comprised in a lysate being administered, the lysate may be free of whole cells. The lysate may in some embodiments comprise a solvent, such as water.

In particular embodiments, the glycoengineered tumor cell fragment comprises or consists of a cell membrane, e.g. a cell membrane of a or originating from a tumor cell. In such embodiments, the cell membrane comprises the tumor cell surface, which may be considered as a tumor cell membrane surface. The tumor cell membrane surface then preferably comprises the one or more carbohydrate antigen moieties. The carbohydrate antigen moieties may thus be outer surface carbohydrate antigen moieties. It is understood that a glycoengineered tumor cell fragment comprising a tumor cell surface does not necessarily mean that the tumor cell fragment has to comprise a whole tumor cell surface, but it can well be the case that the tumor cell fragment comprises only a fragment of a whole tumor cell surface, i.e. a tumor cell surface fragment.

In preferred embodiments, the carbohydrate antigen moiety is chemically, in particular covalently, bound to the tumor cell surface. For example, the carbohydrate moiety may be bound to the tumor cell surface via a urea, i.e. carbamide, group or via a 1,2-aminohydroxyethyl group.

In some embodiments, the tumor cell surface comprises different carbohydrate antigen moieties, in particular at least two, at least three, at least four or at least five, different carbohydrate antigen moieties.

In some embodiments, the glycoengineered tumor cell or a glycoengineered tumor cell fragment is a personalized glycoengineered tumor cell or a glycoengineered tumor cell fragment. Such a personalized glycoengineered tumor cell or a glycoengineered tumor cell fragment comprises carbohydrate antigen moieties being configured to bind to endogenous anti-carbohydrate antibodies present in the specific subject. The glycoengineered tumor cell or a glycoengineered tumor cell fragment is typically a personalized cancer vaccine. A personalized glycoengineered tumor cell or a glycoengineered tumor cell fragment can be obtained as described herein, e.g. for example by the method described with respect to the third aspect. Since every subject has a specific and individual anti-carbohydrate antibody composition (for example, different concentrations of different antibodies) a personalized glycoengineered tumor cell or a glycoengineered tumor cell fragment improves the treatment efficiency. Therefore, determining and selecting endogenous anti-carbohydrate antibodies of the subject and identifying the one or more carbohydrate antigen moieties corresponding to the selected endogenous anti-carbohydrate antibodies can provide for each patient a specific and thus personalized treatment.

Typically, a glycoengineered tumor cell is a tumor cell which has been artificially, preferably in-vitro, modified, in particular by chemical ligation, enzymatic glycosylation or glycolipid cell membrane insertion.

As the skilled person understands, a carbohydrate antigen moiety is a moiety which can interact, respectively bind with a corresponding antibody. Thus, the carbohydrate antigen moiety may be a carbohydrate epitope. For example, an α-gal epitope may be bound by a corresponding anti-Gal antibody. In preferred embodiments, the carbohydrate antigen moiety corresponds to an anti-carbohydrate antibody being naturally present in the subject. Particularly, at least one carbohydrate antigen corresponds to a natural anti-carbohydrate antibody of IgG isotype present in the circulatory system of the subject and/or at least one carbohydrate antigen corresponds to a natural anti-carbohydrate antibody of IgM isotype present in the circulatory system of the subject and/or at least one carbohydrate antigen corresponds to a natural anti-carbohydrate antibody of both IgG and IgM isotypes present in the circulatory system of the subject.

The glycoengineered tumor cell or the glycoengineered tumor cell fragment represents a personalized cancer vaccine which augments immunogenicity of the adaptive immune system towards cancer cells. Anti-carbohydrate antibodies naturally present in a subject recognize the carbohydrate antigens present on any carriers, proteins, linkers, or the modified cancer cell membranes. Previous studies have suggested or shown that natural IgG anti-carbohydrate antibodies can be exploited as endogenous adjuvants enhancing immunogenicity towards cancer vaccines, by processing such vaccines through antigen-presenting cells (APCs) (LaTemple et al. 1996, Galili and LaTemple 1997, doi 10.1016/s0167-5699(97)80024-2). In vivo targeting of a vaccine dose towards MHC- or HLA-dependent antigen presentation can be achieved, for instance, by complexing with various subclasses of IgG molecules. This is because APCs express Fcγ receptors that can effectively interact with the Fc portion of opsonizing IgG antibodies and mediate the uptake of IgG-complexed antigens, such as those ligated or inserted antigenic carbohydrate moieties on the modified cancer cell membrane. This way, not only carbohydrate antigens binding to IgG antibodies will be internalized into APCs, but endogenously expressed tumor antigens also present on the opsonized tumor cell membranes. Binding of antigen-antibody immune complexes to Fcγ receptors expressed on APCs such as dendritic cells (DCs) induces effective maturation of the DCs, resulting in effective cross-presentation of the antigenic peptides on MHC class I molecules to CD8+ cytotoxic T cells; the presentation of immunogenic peptides on MHC class II molecules can also be expected and will lead to the activation of CD4+ helper T cells. An immune response will therefore also be triggered against potentially uncharacterized tumor antigens present on the cancer cells and eventually long-lasting anti-tumor antibodies will be mounted in the subject. Autoimmune responses to normal antigens on non-cancer cells are not expected (Galili 2004, doi 10.1007/s00262-004-0524-x). In addition to the Fcγ receptor-related mechanisms, also polymeric IgM-related immune responses may be enhanced through the activation of the complement system. Collectively, both humoral and cellular immune responses are augmented by the inventive personalized cancer vaccine.

A subject as used herein is typically a mammal, such as a human being or an animal and further a subject suffering from cancer.

As used herein, if the term “consisting” is used then no further features are present in the product apart from the ones following said wording. In contrast, if the term “comprising” is used it includes those features following this term, but that it does not exclude the presence of other features, as long as they do not render the claim unworkable.

As used herein, neoplasm is defined as cells that grow and multiply forming a mass of tissue, collectively called a tumor. The term tumor refers to a form of cells that grow and divide aberrantly (i.e., more than they should) or do not die normally when they should. If benign, they are referred as noncancerous, if malignant, tumors are considered cancerous. Cancerous tumors may invade neighboring tissues and spread throughout the body, a process called metastasis. Benign tumors, however, generally do not invade neighboring tissues and do not spread throughout the body. Neoplasms can be divided into those of the blood and blood-forming tissue (leukemias) and solid tumors. Solid tumors are an abnormal mass of tissue that normally does not contain cysts or liquid areas. Examples of solid tumors are sarcomas, carcinomas, and lymphomas. Further types of solid tumors are defined based on the type of cells that form them.

Sarcomas are a type of cancer that progresses in bone or in soft tissues of the body, such as cartilage, fat, muscle, blood vessels, fibrous tissue, or other connective or supportive tissue. Accordingly for instance, osteosarcoma forms in bone, liposarcoma forms in fat, and rhabdomyosarcoma forms in muscle tissue. Carcinomas are cancers that originate in the skin or in tissues that line or cover internal organs. Tumors that begin in cells of the immune system are lymphomas. Hodgkin lymphoma is marked by the presence of Reed-Sternberg cells, while non-Hodgkin lymphomas include a large and diverse group of cancers of immune cells. This latter can be further subdivided into slow-growing (indolent) or fast-growing (aggressive) non-Hodgkin lymphomas.

The glycoengineered tumor cell or a glycoengineered tumor cell fragment of the present invention is applicable to almost all types of cancerous neoplasms. Representative examples of cancers that may be treated in the context of the present invention based on their tissue specificity include esophageal cancer, oropharyngeal cancer, stomach cancer, colon cancer, rectal cancer, cancer of the anal region liver cancer, pancreatic cancer, cancer of the head and the neck, lung cancer, non-melanoma skin cancer, melanoma, cancer of the bladder, renal cancer, prostate cancer, ovarian cancer, uterine cancer, cervical cancer, breast cancer, Hodgkin and non-Hodgkin lymphomas, cancer of the endocrine system, cancer of the thyroid gland, bone cancer, chronic or acute leukemias, neoplasm of the central nervous system (CNS), glioma, etc., in certain examples lung cancer. The cancer patients are in some embodiments suffering from a solid tumor. The cancer patients are in some embodiments suffering from a hematological malignancy. The cancer patients may be suffering from an advanced cancer, including metastatic solid cancers or cancers associated with high risk of relapse.

In some embodiments, the treatment and/or prevention of cancer includes the administration of the glycoengineered tumor cell or tumor cell fragment to the subject, in particular by oral or parenteral administration, preferably subcutaneous, intramuscular or intravenous administration.

In some embodiments, the glycoengineered tumor cell or tumor cell fragment is an endogenous tumor cell of the subject, i.e. it is a cell or cell fragment which has been obtained from the subject. In some embodiments, the tumor cell or tumor cell fragment originates from the subject. For example, a tumor cell or tumor cell fragment can be obtained from the subject and is then modified, particularly in-vitro modified, to produce the glycoengineered tumor cell or glycoengineered tumor cell fragment. Modification of the tumor cell or tumor cell fragment can be achieved by a chemical ligation, enzymatic glycosylation reaction or glycolipid cell membrane insertion. For example, by reacting an epoxide group linked to a carbohydrate moiety with a free amino group of the tumor cell surface resulting in the formation of a 1,2-aminohydroxyethyl moiety.

In some embodiments, the one or more carbohydrate antigen moieties correspond to endogenous anti-carbohydrate antibodies of the subject, i.e. the subject treated with the glycoengineered tumor cell or a glycoengineered tumor cell fragment. As an individual subject typically has an individual anti-carbohydrate antibody composition, such embodiments allow to provide a highly effective personalized immune response. It is clear to the skilled person that a carbohydrate antigen moiety which corresponds to a specific antibody is a moiety, respectively has an epitope, which can be bound, e.g. selectively bound, by a corresponding anti-carbohydrate antibody. For example, an α-gal epitope may be bound by a corresponding anti-Gal antibody.

In particular, natural anti-carbohydrate antibodies denote herein but not restrict to of IgM and IgG isotypes.

In some embodiments, the one or more carbohydrate antigen moieties have an equilibrium constant K_{d}=k_{off}/kₒₙ with the endogenous anti-carbohydrate antibodies of less than 1 mM, in particular less than 1 μM. kₒₙ is the rate constant characterizing how fast the antibody binds to the antigen and k_{off} is the rate constant characterizing how fast the antibody dissociates from the antigen.

In some embodiments, the treatment and/or prevention of cancer in the subject comprises determining and selecting endogenous anti-carbohydrate antibodies of the subject and identifying the one or more carbohydrate antigen moieties corresponding to the selected endogenous anti-carbohydrate antibodies. Thus, at first the subject may be screened for its available anti-carbohydrate antibodies. Subsequently, the most promising carbohydrate antigen moieties are selected such that they correspond to the selected endogenous anti-carbohydrate antibodies, particularly the endogenous anti-carbohydrate antibodies which may be most abundant in the subject to be treated. For example, the at least one, at least two, three, four or five carbohydrate antigen moieties are selected which bind to the at least two, three, four or five most abundant endogenous anti-carbohydrate antibodies present in the subject and/or sample.

In some embodiments, the carbohydrate antigen moieties are glycan moieties. In particular, the glycan moieties may be selected from Rha-, ManNAc-, GlcNAc-, Galβ1-4Glc-, Galβ1-6Galβ1-4Glc-, GlcNAcβ1-2Galβ1-3GalNAc-, GlcNAcβ1-6(GlcNAcβ1-4)GalNAc-, Galα1-6Glc-, Galα1-3GlcNAc-, GalNAcα1-3GalNAc-, GlcNAcβ1-4(6-O-Su)GlcNAc-, Galβ1-3GlcNAcα1-6Galβ1-4GlcNAc-, Galβ1-3GlcNAcα1-3Galβ1-3GlcNAc-, Galα1-4GlcNAc-, 6-Bn-Galα1-4(6-Bn)GlcNAc-, GlcNAcβ1-4-MurNAc-, GlcNAcβ1-4Mur-, GalNAcα1-3GalNAcβ1-3Gal-, Galα1-4GlcNAcβ1-3Galβ1-4GlcNAc-, Galβ1-3GlcN(Fm)β1-3Galβ1-4GlcNAc-, GlcNAcβ1-4GlcNAc-, Galα1-2Gal-, Galα1-3Galβ1-4Glc-, GlcNAcα1-3GalNAc-, Neu5Acα2-8Neu5Ac-, Fucα1-2Galβ1-4Glc-, Neu5Acα2-3Galβ1-4Glc-, GlcNAcβ1-4Galβ1-4GlcNAc-, Neu5Acα2-8Neu5Acα2-4Neu5Ac-, Galβ1-3GlcNAcβ1-3Galβ1-4Glc-, Galβ1-4GlcNAcβ1-3Galβ1-4Glc-, Galα1-3Galβ1-4GlcNAcβ1-3Galβ1-4Glc-, Arafβ1-2Arafα1-5(Arafβ1-2Arafα1-3)Arafα1-5Araf-, Neu5Acα2-8Neu5Acα2-4Neu5Acα2-6GlcNAcβ1-6GlcNAc-, Fucα1-4GlcNAc-, GalNAcα1-3GalNAcβ1-3Galα1-4Galβ1-4Glc-, GlcNAcα1-3Galβ1-4GlcNAc-, 3-O-Su-GlcNAc-, 6-O-Su-Gal-, GalNAcα1-3Gal-, GlcNAcβ1-3GalNAc-, (6-O-Bn-Galβ1)-3GlcNAc-, Galα1-3GalNAc(fur)-, 4,6-O-Su2-Galβ1-4GlcNAc-, GlcNAcβ1-6(GlcNAcβ1-3)GalNAc-, Galβ1-4Galβ1-4GlcNAc-, GalNAcα1-3Galβ1-4GlcNAc-, Galβ1-3GlcNAcα1-3Galβ1-4GlcNAc-, Galβ1-3GlcN(Fm)β1-3Galβ1-3GlcNAc-, Galβ1-3GalNAcβ1-3Galα1-4Galβ1-4Glc-, Galβ1-3GalNAc(fur)-, Fucα1-3GlcNAc-, Fucβ1-3GlcNAc-, Galβ1-3GlcNAc-, GlcNAcβ1-6GalNAc-, 3-O-Su-Galβ1-3GlcNAc-, 6-O-Su-Galβ1-3GlcNAc-, GlcAβ1-3GlcNAc-, 3,4-O-Su2-GalNAcβ1-4GlcNAc-, Galβ1-4GlcNAcβ1-6GalNAc-, and GlcNAcβ1-4Galβ1-4GlcNAc-.

In some embodiments, the carbohydrate antigen moieties are glycan moieties, with the proviso that the carbohydrate antigen moieties are not Gal, in particular α-Gal, and that the carbohydrate antigen moieties are not N-propionylated polysialic acid-polysialic glycans.

In some embodiments, the glycoengineered tumor cell is an inactivated tumor cell, e.g. the tumor cell cannot divide further. Inactivation can for example be achieved by irradiation.

In some embodiment the use comprises, the method, in particular the in-vitro method, for producing a glycoengineered tumor cell or glycoengineered tumor cell fragment, as described herein, in particular with respect to the third aspect of the invention.

A second aspect of the invention relates to a pharmaceutical composition for use in treatment and/or prevention of cancer in a subject. The pharmaceutical composition comprises one or more of the glycoengineered tumor cell or one or more glycoengineered tumor cell fragment according to any of the embodiments described herein, in particular with respect to the first aspect of the invention. If more than one glycoengineered tumor cells or glycoengineered tumor cell fragments are used, they may in some embodiments comprise different carbohydrate antigen moieties. However, it is clear the pharmaceutical composition comprises typically at least 10³, in particular at least 10⁴, in particular at least 10⁶, in particular at least 10⁶ glycoengineered tumor cells or glycoengineered tumor cell fragments of the same type, i.e. having the same carbohydrate antigen moieties. Furthermore, the pharmaceutical composition may comprise a pharmaceutical carrier, such as a pharmaceutically acceptable carrier, and/or an adjuvant, and/or a buffer and/or a solvent. In some embodiments, the glycoengineered tumor cell or tumor cell fragment is present in a pharmaceutical acceptable amount. The term “therapeutically-effective amount” as used herein refers to the amount of a glycoengineered tumor cell or tumor cell fragment of the present disclosure which is effective for producing some desired therapeutic effect.

Suitable buffers are for example TRIS (tris(hydroxymethyl)methylamine), TRIS-HCl (tris(hydroxymethyl)methylamine-HCl), HEPES (4-2-hydroxyethyl-1-piperazineethanesulfonic acid), phosphate buffer (e.g. PBS; HBSS, mixture of Na2HPO4 and KH2PO4; mixture of Na2HPO4 and NaH2PO4), TEA (triethanolamine), EPPS (N-(2-Hydroxyethyl)-piperazine-N′-3-propanesulfonic acid), TRICINE (N-[Tris(hydroxymethyl)-methyl]-glycine) and bicarbonate buffers are particularly appropriate for maintaining a physiological or slightly basic pH (e.g. from approximately pH 7 to approximately pH 9). The buffer (e.g. TRIS-HCl) is preferably present at a concentration of 10 to 50 mM. It might be beneficial to also include a monovalent salt to ensure an appropriate osmotic pressure. Said monovalent salt may notably be NaCl or KCl.

Pharmaceutical compositions may be in unit dose form containing a predetermined amount of a glycoengineered tumor cell or glycoengineered tumor cell fragment of the present disclosure per unit dose. Such a unit may contain a therapeutically effective dose of a glycoengineered tumor cell or tumor cell fragment of the present disclosure or salt thereof or a fraction of a therapeutically effective dose such that multiple unit dosage forms might be administered at a given time to achieve the desired therapeutically effective dose. In some embodiments, unit dosage formulations are those containing a daily dose or sub-dose, or an appropriate fraction thereof, of a glycoengineered tumor cell or glycoengineered tumor cell fragment of the present disclosure.

The phrase “pharmaceutically carrier” as used herein means a pharmaceutically-acceptable material, composition or vehicle, such as a liquid or solid filler, diluent, excipient, manufacturing aid, or solvent encapsulating material, particularly involved in carrying or transporting the subject glycoengineered tumor cell or glycoengineered tumor cell fragment from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical compositions.

Such compositions may contain components conventional in pharmaceutical preparations, e.g. wetting agents, emulsifiers and lubricants, such as sodium lauryl sulfate and magnesium stearate, as well as coloring agents, release agents, coating agents, sweetening, flavoring and perfuming agents, preservatives and antioxidants, pH modifiers, bulking agents, and additional active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Such compositions may be prepared by any method known in the art, for example, by bringing into association the glycoengineered tumor cell or glycoengineered tumor cell fragment with one or more carriers and/or excipients. Excipients that may be used in the preparation of the pharmaceutical compositions may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

Liquid dosage forms of the glycoengineered tumor cell or glycoengineered tumor cell fragment of the present disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The dosage levels of a glycoengineered tumor cell or glycoengineered tumor cell fragment of the present disclosure in the pharmaceutical compositions of the present disclosure may be adjusted in order to obtain an amount of a glycoengineered tumor cell or glycoengineered tumor cell fragment of the present disclosure which is effective to achieve the desired therapeutic response for a particular patient, composition, and mode of administration, without being deleterious to the patient. The dosage of choice will depend upon a variety of factors including the nature of the particular glycoengineered tumor cell or glycoengineered tumor cell fragment of the present disclosure used, the route of administration, the time of administration, the rate of excretion or metabolism of the particular glycoengineered tumor cell or glycoengineered tumor cell fragment used, the rate and extent of absorption, the duration of the treatment, other drugs, compounds and/or materials used in combination with the particular glycoengineered tumor cell or glycoengineered tumor cell fragment, the age, sex, weight, condition, general health and prior medical history of the patient being treated, and like factors well known in the medical arts. A medical practitioner having ordinary skill in the art can readily determine and prescribe the effective amount of the pharmaceutical composition required.

In some embodiments, a suitable daily dose of a glycoengineered tumor cell or glycoengineered tumor cell fragment of the present disclosure will be that amount of the glycoengineered tumor cell or glycoengineered tumor cell fragment which is the lowest dose effective to produce a therapeutic effect. Such an effective dose will generally depend upon the factors described above.

The effective dose of a glycoengineered tumor cell or glycoengineered tumor cell fragment of the present disclosure may be administered as two, three, four, five, six or more sub-doses administered separately at appropriate intervals throughout a specified period (per day or per week or per month), optionally, in unit dosage forms. In some embodiments, dosing also depends on factors as indicated above, e.g. on the administration, and can be readily arrived at by one skilled in medicine or the pharmacy art.

A third aspect of the present invention relates to a method, in particular an in-vitro method, for producing a glycoengineered tumor cell or tumor cell fragment, in particular a glycoengineered tumor cell or tumor cell fragment according to any of the embodiments as described herein, in particular with respect to the first aspect of the invention. The method comprises the steps
a. Providing a sample comprising anti-carbohydrate antibodies of a subject. This step may in some embodiments exclude the actual sample collection from the subject. However, this step may in some embodiments be preceded by a collection step in which sample is collected from the subject, i.e. patient. This may for example be achieved by withdrawing and processing a blood sample from the subject according to standard medical procedures. Taking a blood biopsy, preparing a serum sample, and the actual assessment of natural anti-carbohydrate antibodies therein are not necessarily taking place at the same or similar time. For example, serum samples may be prepared shortly after blood withdrawal and stored for long-term according to standard guidelines, and the anti-carbohydrate antibodies contained therein may be analyzed at a later stage.
b. In-vitro determining and selecting endogenous anti-carbohydrate antibodies of the subject and identifying the one or more carbohydrate antigen moieties corresponding to the selected endogenous anti-carbohydrate antibodies.
c. In-vitro glycoengineering a tumor cell or tumor cell fragment by providing a tumor cell surface of the tumor cell or tumor cell fragment with the identified one or more carbohydrate antigen moieties to produce the glycoengineered tumor cell or glycoengineered tumor cell fragment.

It is understood that the anti-carbohydrate antibodies of the subject comprised in the sample being provided in step a. are endogenous anti-carbohydrate antibodies. Further it is understood that the carbohydrate antigen moieties identified in step b. are configured to bind the selected endogenous anti-carbohydrate antibodies, that is they correspond to the selected endogenous anti-carbohydrate antibodies. The method according to the invention allows to provide a personalized glycoengineered tumor cell or tumor cell fragment which can be used as a personalized cancer vaccine.

It is further understood that the tumor cell or tumor cell fragment of step c. originates or is obtained from the subject from which the sample has been provided in step a.

In some embodiments, the method further comprises step d:
d. Disaggregating, in particular lysing, the glycoengineered tumor cell obtained in step c. to provide the glycoengineered tumor cell fragment. The glycoengineered tumor cell fragment may for example be comprised in a lysate. Step d. represents in some embodiments an alternative access for the generation of the glycoengineered tumor cell fragment. While in some embodiments as described above, a yet non-engineered tumor cell fragment can be provided in step c. and is in-vitro glycoengineered to produce the glycoengineered tumor cell fragment, a tumor cell is in some embodiments first glycoengineered in step c. to provide a glycoengineered tumor cell which is then disaggregated according to step d.

In some embodiments, the glycoengineered tumor cell fragment obtained in step d. is purified and/or isolated after step d., in particular separated from other cellular components generated during step d. It is understood that the purified and/or isolated glycoengineered tumor cell fragment comprises the one or more carbohydrate antigen moieties. Preferably and as mentioned herein above, the glycoengineered tumor cell fragment purified and/or isolated after step d. comprises or consists of a cell membrane. In such embodiments, the cell membrane comprises the tumor cell surface, which may be considered as a tumor cell membrane surface. The tumor cell membrane surface then preferably comprises the one or more carbohydrate antigen moieties. The carbohydrate antigen moieties may thus be outer surface carbohydrate antigen moieties.

In some embodiments, the tumor cell or tumor cell fragment is inactivated before, during or after step c. This means that the tumor cell or tumor cell fragment can after inactivation not divide further. Inactivation can for example be achieved by irradiation.

In some embodiments, step c. comprises a chemical ligation, enzymatic glycosylation or glycolipid cell membrane insertion. Glycoengineering of a tumor cell or tumor cell fragment may for example in this or any other embodiment as described herein, and in particular in the examples presented herein, be performed as described in Dekany et al U.S. Pat. No. 8,785,594 (U.S. Ser. No. 12/733,672), which is included herein by reference in its entirety and in particular regarding its examples 1-21 and the claims. Preferably, the chemical ligation comprises the formation of a urea, i.e. carbamide, group or a 1,2-aminohydroxyethyl between the tumor cell or tumor cell fragment and the one or more carbohydrate antigen moiety, respectively a derivative thereof. A suitable derivative thereof may be a derivative having a ligation site being configured to undergo a ligation reaction with a chemical functional group of the tumor cell surface, such as a free amino group. For example, the derivative of the carbohydrate antigen moiety comprises a carbamate being linked to the carbohydrate moiety, in particular a cyclic, preferably strained, carbamate, which then reacts with a free amine of the tumor cell surface.

For enzymatic conjugation, the tumor cell or tumor cell fragment is incubated with the selected carbohydrate antigen moiety donor(s) and the corresponding enzymes capable of transferring the carbohydrate antigen moiety onto the cell surface of the tumor cell or tumor cell fragment in a way that the desired immunogenic carbohydrate structure is formed on the cell surface of the tumor cell or tumor cell fragment. Processing of glycoproteins may include a step of removal of a monosaccharide performed by a suitable enzyme, and a step of conjugating one of the antigens to a glycan of the glycoprotein with another suitable enzyme at the position where the other glycan has been removed. Processing of glycoproteins may also potentially include only a single step of conjugating one of the carbohydrate antigen moieties to an already existing glycan of the glycoprotein with another suitable enzyme. After incubation of the tumor cell or tumor cell fragment with the enzymes needed, a washing step may usually be followed by removing the residual enzymes and antigens. The approach relating to the insertion of glycolipids into the cell membrane takes advantage of the natural tendency of lipids to insert themselves into lipid bilayer such as cell membranes. For this approach, different glycolipids are prepared and used, each having the selected carbohydrate antigen moiety already displayed on the glycosyl moiety of the glycolipid. The lipid portion of the glycolipid may be e.g., cholesterol, phospholipid or ceramide. Extracted glycolipids tend to dissolve in water or suitable buffers forming micelles in which the hydrophobic portion of the glycolipid (i.e., the lipid tail) is in the core of the micelle whereas the hydrophilic glycosyl moiety protrudes into the aqueous surroundings. The tumor cell or tumor cell fragment is incubated with the glycolipid micelles for e.g., 2-3 h at 37° C., which should result in spontaneous insertion of these glycolipids into the cell membrane, since the hydrophobic lipid tail of the glycolipid is energetically more stable when surrounded by the phospholipids of the lipid bilayer of the cell membrane than when it is surrounded by water molecules in the micelle.

In some embodiments, the one or more carbohydrate antigen moieties have an equilibrium constant K_{d}=k_{off}/kₒₙ with the endogenous anti-carbohydrate antibodies of less than 1 mM, in particular less than 1 μM. kₒₙ is the rate constant characterizing how fast the antibody binds to the antigen and k_{off} is the rate constant characterizing how fast the antibody dissociates from the antigen.

In some embodiments, step b. comprises the determination of isotype- and subclass-specific anti-carbohydrate antibody titers provided by the sample. In certain embodiments, the endogenous anti-carbohydrate antibodies are selected based on the presence or absence of isotype- and subclass-specific antibody titers.

In some embodiments, step b. is preferably performed using printed glycan array (PGA) technology. PGA technology, also referred to herein as carbohydrate (antigen) arrays or glycan (antigen) arrays, are composed of a repertoire of different glycans immobilized on a solid support in a spatially defined arrangement and have therefore the potential to map out carbohydrate interactions in a high-throughput manner analyzing the same and occasionally limited amount of patient serum sample. Glycan array may be performed according to any standard technique known in the art (Huflejt et al. 2009, doi 10.1016/j.molimm.2009.06.010; Oyelaran et al. 2009, doi 10.1021/pr900515y; Bello-Gil et al. 2017, doi 10.3389/fimmu.2017.01449). The glycans may each be printed in several replicates, depending on the background signal noise of an average serum sample. Glycans present on the glycan array are referred to herein as saccharide or oligosaccharide, in free form (before immobilization) or attached to another molecule, or any natural or non-natural compound that mimic the physical, structural, and chemical characteristics including the biological activities of natural or engineered functions of saccharides, and any combinations thereof. The glycans used for the glycan arrays may be or comprise antigenic carbohydrates. The glycans used for the glycan arrays may be naturally occurring glycans or may be obtained from libraries generated from natural glycans or from chemically synthesized libraries.

The glycans may be selected from blood group antigens, some of the most frequently occurring terminal oligosaccharides and core motifs of mammalian N- and O-linked glycoproteins and glycolipids, tumor-associated tumor carbohydrate antigens, carbohydrate tumor neoantigens and/or polysaccharides from pathogenic bacteria.

Detection of isotype-specific serum antibodies bound to the glycans of the array is typically performed with a single or a combination of fluorescently-tagged secondary antibodies, followed by image processing and data analysis as described (Huflejt et al. 2009, doi 10.1016/j.molimm.2009.06.010; Oyelaran et al. 2009, doi 10.1021/pr900515y; Bello-Gil et al. 2017, doi 10.3389/fimmu.2017.01449). In the preferred embodiment of, for example, IgG and IgM anti-carbohydrate antibody detection, anti-human IgG and anti-human IgM antibodies tagged with isotype-specific fluorophores are added simultaneously to the serum bound glycans on the glycan array; this enables the parallel detection of patient-specific anti-carbohydrate IgM and IgG isotypes present in serum. After data analysis (e.g., performed with a ScanArray® Express Microarray Analysis system (PerkinElmer, Waltham, MA, USA)), the binding signals may be expressed in any readout-specific arbitrary units, such as relative fluorescence units (RFU) as median±median absolute deviation (MAD). Additionally, a standard spot/value on each glycan array shall be used for signal normalization to minimize variance typically occurring between various samples as well as runs. A threshold for the identification of background signals is usually also determined.

An enzyme linked immunosorbent assay (ELISA) may additionally be used as an alternative or as a validation tool for such an antibody-based detection assay. An ELISA assay allows for the quantitative validation of the presence or absence of total isotype-specified antibodies, such as natural anti-carbohydrate antibodies, in a biological sample, such as serum purification following a fluid biopsy. In a typical form of this assay, the antigens to be tested, such as carbohydrate epitopes, are attached typically to a microtiter plate surface directly or through ligation to a typical carrier protein bound to said surface. The requirement for such a carrier protein is that the serum sample to be analyzed shall contain no antibodies targeting the carrier protein itself so during incubation with the patient sample, its serum antibodies are allowed to specifically bind the respective antigen. An example for such carrier proteins is the keyhole limpet hemocyanin, a large, multiunit, oxygen-carrying metalloprotein that is found in the hemolymph of the giant keyhole limpet, Megathura crenulate. After serum incubation, bound patient antibodies are labelled in a subsequent step with an anti-human isotype specific antibody which is additionally linked to an enzyme. In the final step, a substance containing the enzyme's substrate can be added which after the enzymatic reaction produces a detectable signal, most commonly a color change. Such results can be read out with a suitable spectrophotometer, e.g., a VICTOR multilabel or multimode plate reader (PerkinElmer, Waltham, MA, USA). Binding signals, in combination with a sample titration approach, may eventually be expressed as concentration values valid for the particular patient serum. Additionally, a standard antigen with a standard antibody combination shall be used for signal normalization to support the qualitative analysis. A threshold for the identification of background signals is usually also determined.

In some embodiments, the tumor cell or tumor cell fragment being glycoengineered in step c. is an endogenous tumor cell of the subject, i.e. it is a cell or cell fragment which has been obtained from the subject. Additionally or alternatively, the tumor cell or tumor cell fragment originates from the subject. It is understood that the subject is the same from which the sample in step a. originates.

A fourth aspect of the invention relates to a method of treatment of a subject comprising the administration of a glycoengineered tumor cell or glycoengineered tumor cell fragment or a pharmaceutical composition according to any of the embodiments as described herein, in particular with respect to the first aspect or the second aspect of the invention, to the subject or the administration of a pharmaceutical composition according to any of the embodiments as described herein, in particular with respect to the second aspect of the invention, to the subject. In some embodiments, the method of treatment, in particular, the administration, is preceded by the method for producing a glycoengineered tumor cell or tumor cell fragment according to any of the embodiments as described herein, in particular for the third aspect of the invention.

In some embodiments, the tumor cell or tumor cell fragment being glycoengineered, in particular in step c. of the method of the third aspect of the invention, is obtained by biopsy, in particular by routine clinical biopsy of the patient. A cancer sample obtained by biopsy includes e.g., blood or a tissue sample obtained from the primary tumor or from tumor metastases or any other related sample containing cancerous cells. The tumor cell or tumor cell fragment can be isolated according to any technique known in the art. For example, tumor cell or tumor cell fragment of solid tumors are typically obtained from surgery, which may further be processed through e.g., tissue dissociation, enzymatic digestion, gradient separation, red blood cell lysis, sterilization, filtering, or homogenization.

Preferably, the method or use according to the invention may be carried out in conjunction with surgery. For example, the glycoengineered tumor cell or tumor cell fragment, respectively the pharmaceutical composition, may be administered after partial or total surgical resection of the tumor or the cancerous tissue itself to minimize the possibility for relapse or metastasis (e.g., by local application within the excised zone).

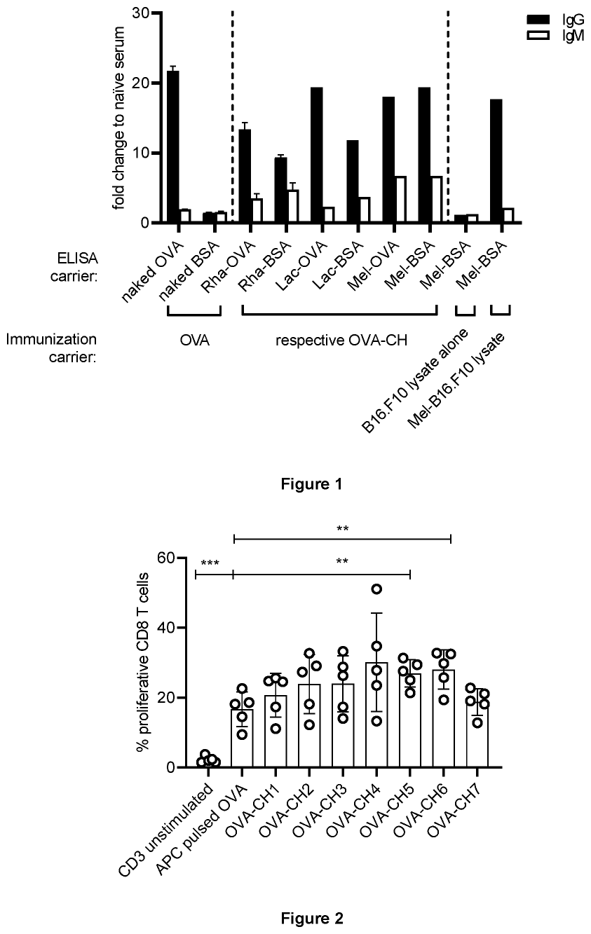

FIG. 1 shows comparative data of C57BL/6 mice treated with the indicated samples. In the experiments, C57BL/6 mice were immunized subcutaneously with either 50 μg OVA (ovalbumin) alone or 50 μg carbohydrate-ligated ovalbumin in PBS (phosphate buffered saline) (OVA-CH; CH: Carbohydrate; Rha: rhamnose; Lac: lactose; Mel: melibiose). Alternatively, mice were immunized with lysates of either 4×10⁵ non-ligated B116.F10 cells or 4×10⁵ melibiose-ligated B16.F10 murine melanoma cells. For the latter, cells were incubated with 40 mM ligating probe in HBSS (Hank's balanced salt solution) at 37° C. for 3 hours while mildly agitated. Subsequently, both ligated and non-ligated cells were washed with PBS, processed through 6 rounds of a freeze-thaw cycle (5 min liquid N₂, 5 min 37° C. water bath) and sonicated for 2 min on ice. For immunization, OVA solutions were mixed 1:1 with CFA (Complete Freund's Adjuvant or B16 tumor cell lysates were mixed 1:1 with adjuvant mix of MPL/Alum (monophosphoryl lipid A and Alum). OVA-immunized animals received a booster shot in which the same OVA solutions were mixed 1:1 with IFA (Incomplete Freund's Adjuvant) in a similar fashion as for the primary immunization. As an endpoint readout, blood was withdrawn from the animals, serum samples were prepared and stored at −20° C. until further analysis. Raised anti-carbohydrate antibody levels as a mean of the immunogenic potency of both the ligated protein vaccine as well as the ligated tumor vaccine were assessed by the analysis of 1:20 diluted murine sera. As ELISA carriers, either naked OVA or carbohydrate-ligated OVA were used indicating combined immunization efficacy of OVA/OVA-CH epitopes (i.e. against OVA and CH); or naked bovine serum albumin (BSA) or carbohydrate-ligated bovine serum albumin (BSA) were used indicating the single immunization efficacy of CH epitopes alone.

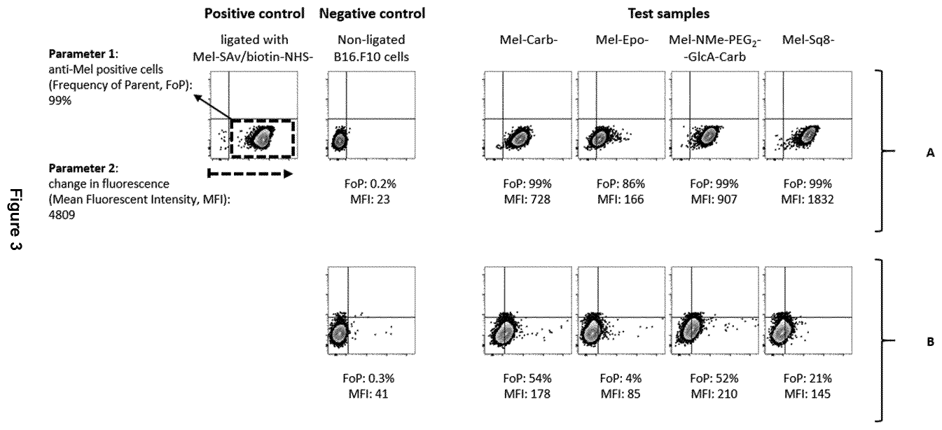

FIG. 2 shows in vitro data resembling the capacity of antigen presenting cells (APC) to boost T cell immunity in the absence or presence of immunogenic carbohydrates and their corresponding human natural anticarbohydrate antibodies. C57BL/6 mice were tape-stripped on the ear skin and epicutaneously immunized with 40 μg ovalbumin (OVA, InvivoGen) in combination with 5 μg CpG ODN, type B (InvivoGen). After seven days, spleens were harvested, CD3+ splenocytes sorted by MACS (Pan T cell isolation kit II, Miltenyi) (→“CD3 unstimulated”). In parallel, another set of splenocytes were harvested from age-matched naïve C57BL/6 animals, depleted for Thy1.2+ cells by MACS (CD90.2 microbeads, Miltenyi) and irradiated for 1 min (160 KV, 6.3 mA, 19,57 gray/min, no copper filter). This latter mix of antigen-presenting cells was then pulsed with 100 nM OVA (→“APC pulsed OVA”) or its carbohydrate-ligated form (→“OVA-CH1-7”) for 3 hours in the presence of 2.5 μg/ml serum-purified human total IgG mixed with 2.5 μg/ml total IgM. In vivo OVA-primed CD3 T cells were permanently labelled with a cell proliferation dye (eFluor 670, eBioscience) and then co-cultured with ex vivo OVA-pulsed Thy1-negative APC for 72 hours. As an endpoint readout, T cell proliferation was assessed by FACS. As can be seen, all cells pulsed with carbohydrate-ligated OVA show elevated potencies of T-cells to proliferate as compared to the cells pulsed with OVA only, thereby indicating that carbohydrate antigens can improve the immune response. Data is represented as T cell co-cultures from individual mice, shown as mean±±SD. Statistical significance was analyzed via unpaired t-test (**p>0.01, ***p>0.001). Carbohydrates ligated onto OVA were tested as follows: OVA-CH1, OVA-rhamnose; OVA-CH2, OVA-rhamnose; OVA-CH3, OVA-rhamnose; OVA-CH4, OVA-lactose; OVA-CH5, OVA-lactose; OVA-CH6, OVA-lactose; OVA-CH7, OVA-lactose. OVA-CH1 to OVA-CH3 are different from each other by the linker linking the OVA and CH moieties. Accordingly, also OVA-CH4 to OVA-CH7 are different from each other by the linker linking the OVA and CH moieties.

FIG. 3 shows FACS plots of different malignant B116.F10 cells ligated with Melibiose (Mel) utilizing various linkages. The top row labelled with “A” shows the results of experiments conducted with murine antibodies for Mel detection and the bottom row labelled with “B” experiments conducted with human antibodies for Mel detection. B16.F10 cells were detached with 2 mM EDTA from running cell cultures (at 37° C. with 5% C02) and washed with sterile Dulbecco's phosphate buffered saline (DPBS) prior manipulation. Positive controls were ligated with 1 mM biotin-(PEG)₄-N-hydroxysuccinimide (biotin-NHS) for 30 min at room-temperature (RT), washed and pelleted 3 times with 100 mM glycine/DPBS to free from non-bound reactive probes and labelled with 3 μg/ml of melibiose-ligated streptavidin (Mel-SAv; SAv was ligated overnight at RT with 25 mM melibiose-epoxide). Test samples were ligated for 3 hours at RT with various Mel probes: either with 20 mM melibiose carbamate (Mel-Carb), melibiose epoxide (Mel-Epo), melibiose N-methyl-(PEG)₂-glycuronic-acid-carbamate (Mel-NMe-PEG₂-GlcA-Carb) or melibiose squarate (Mel-Sq8). Non-ligated negative controls were left untreated but co-incubated with test samples. Test samples and negative control were washed and pelleted 3 times with 100 mM glycine/DPBS. All samples were processed for flow cytometry detection of bound melibiose by using either murine (upper panels) or human anti-Mel antibodies (lower panels). For the detection by murine antibodies, cells were incubated for 1 hour at 4° C. with 1:300 serum dilutions of mice which were subcutaneously immunized with 250 μg melibiose-ligated Keyhole limpet hemocyanin (Mel-KLH; KLH was ligated overnight at RT with 90 mM melibiose-epoxide). For the detection by human antibodies, cells were incubated for 1 hour at 4° C. with 40 μg/ml dilutions of human total IgG fractions which were purified from pooled human healthy donor serums (in.vent Diagnostica; 2.3 mg/ml total protein concentration). Cells were washed and pelleted in ice-cold FACS buffer (1×PBS, 2% BSA). Bound murine and human anti-Mel antibodies were detected by 1:200 dilutions of FITC-conjugated polyclonal rabbit anti-mouse or anti-human IgG F(ab′)2 antibodies (Jackson Immunoresearch; 1.4 mg/ml and 1.5 mg/ml, respectively). All samples were measured on a BD FACSLyric™ analyzer and visualized by using FlowJo (BD). FACS plots of FIG. 3 show the efficiency of cell ligation by determining the frequency of anti-Mel positive cells within the population of total live singlets (parent population) and by determining within that parent population the mean value of fluorescent intensity for the fluorophore conjugated antibody which detects anti-Mel positive cells.

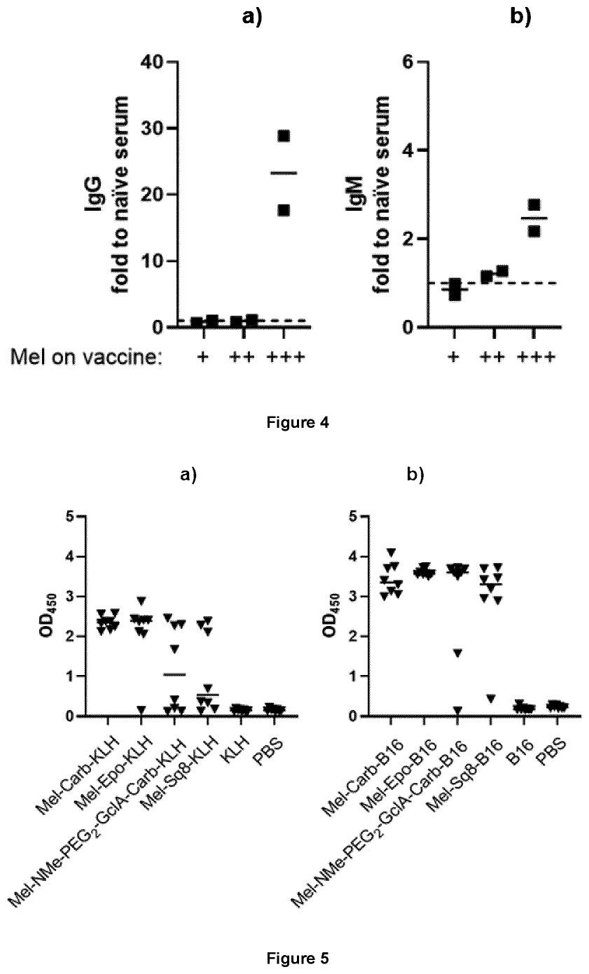

FIG. 4 shows in vivo data for demonstrating the effect of a glycoengineered cell lysate comprising a glycoengineered tumor cell fragment according to the invention on IgG and IgM levels in the subject and thus shows the primary immunogenic effect of such glycoengineered cell lysates. B16.F10 cells were detached with 2 mM EDTA from running cell cultures (at 37° C. with 5% C02) and washed with sterile Dulbecco's phosphate buffered saline (DPBS) prior manipulation. Melibiose carbamate (Mel-Carb) was ligated onto 4×10⁵ B16.F10 cells at 5 mM (+), 20 mM (++) and 40 mM (+++) concentrations in a final reaction volume of 1 ml sterile Hank's balanced salt solution (HBSS, no phenol red) for 3 hours at 37° C. Cells were then washed and pelleted three times by 450×g centrifugation for 5 min at 4° C. with excess volume of sterile DPBS. After the last wash, cells were concentrated in DPBS. Cells were lysed by six freeze-thaw cycles as 5 min in liquid N₂ and 5 min in 37° C. water bath. After the last cycle, lysates were pelleted by 100×g centrifugation for 3 min at 4° C. to pellet large cell debris. Remaining cell lysates were sonicated for 2 min by probe-tip sonicating (Philip Harris). Cell lysates were quick-spined by 100×g centrifugation for 20 sec at 4° C. to pull liquid down. Reaction volumes were adjusted to 75 μl per recipient animal with sterile DPBS. For each recipient 10 μg monophosphoryl Lipid A from Salmonella enterica serotype minnesota Re 595 (Sigma) was mixed with 100 μl Alhydrogel (Invivogen) as adjuvant (MPL/Alum). Ligated cell lysates and adjuvant were mixed together in 1:1 ratio and pipetted up-and-down vigorously for at least 5 min to allow adjuvant to effectively absorb the antigenic mixture. Individual female C57BL/6 recipients (Envigo) for each condition were subcutaneously injected with 50 μl of glycoengineered B16 cell lysate vaccine at each of the two dorsal sites. 4 weeks after immunization, all animals were euthanized, and non-recovery blood samples were withdrawn. Using an enzyme-linked immunosorbent assay (ELISA), MaxiSorp clear flat-bottom 96-well plates were coated with 2 μg/ml of Mel-Carb-ligated ovalbumin (OVA) or Mel-Carb-ligated bovine serum albumin (BSA) overnight at 4° C. Next day, plates were incubated for 1 hour at room-temperate in a sequential order with 1:20 dilution of the experimental serum samples, 1 μg/ml polyclonal rabbit anti-Mo IgG or IgM antibodies (Jackson Immunoresearch), and at 1:5000 dilution of HRP-conjugated goat anti-rabbit IgG detection antibodies (Thermo Fisher Scientific). Between steps, plates were washed three times with excess volume of StartingBlock T20 (TBS) blocking buffer. The presence of serum anti-Mel antibodies were visualized using 1-step Ultra TMB-ELISA Substrate and Stop solutions (Thermo Fisher Scientific) and quantified using a standard plate reader spectrophotometer. The two graphs of FIG. 4 show the average fold increase of anti-Mel IgG (a)) and IgM (b)) antibodies compared to unvaccinated naïve control serum on both OVA and BSA carriers merged.

FIG. 5 shows in vivo data for demonstrating the effect of a glycoengineered cell lysate comprising a glycoengineered tumor cell fragment according to the invention on IgG levels in the subject and thus shows the primary immunogenic effect of such glycoengineered cell lysates. Female C57BL/6 mice (Envigo) were subcutaneously immunized with 250 μg melibiose-ligated (melibiose carbamate (Mel-Carb), melibiose epoxide (Mel-Epo), melibiose N-methyl-(PEG)₂-glycuronic-acid-carbamate (Mel-NMe-PEG₂-GlcA-Carb) or melibiose squarate (Mel-Sq8) Keyhole limpet hemocyanin (KLH), non-ligated KLH or DPBS. Protein ligations were carried out overnight at room-temperature with 90 mM of indicated Mel-ligating probe. For primary and booster immunizations Complete and Incomplete Freund's adjuvants were used, respectively (Sigma Aldrich). 4 weeks after primary immunization animals received melibiose-ligated (melibiose carbamate (Mel-Carb), melibiose epoxide (Mel-Epo), melibiose N-methyl-(PEG)₂-glycuronic-acid-carbamate (Mel-NMe-PEG₂-GlcA-Carb) or melibiose squarate (Mel-Sq8) B16 cell lysate vaccines, non-ligated B16 cell lysate vaccines or DPBS. For both primary and booster vaccinations MPL/Alum adjuvant was used. 4 weeks after primary immunization and 4 weeks after primary vaccination recovery blood samples were withdrawn from the mice and analyzed for the presence of serum anti-Mel IgG antibodies using enzyme-linked immunosorbent assay (ELISA) as described above. The graphs of FIG. 5 show anti-Mel IgG antibody levels of the tested treatment groups. The left graph (a)) shows the response on treatment with Mel on the carrier protein (KLH) and the right graph (b)) the response on treatment with Mel on B16 tumor cell lysate. Each linker chemistry applied for protein and cell ligation was respectively analyzed on the same linker chemistry linking Mel to OVA as carrier for the analysis of samples tested after protein immunization and to BSA as carrier for the analysis of samples tested after lysate vaccination.

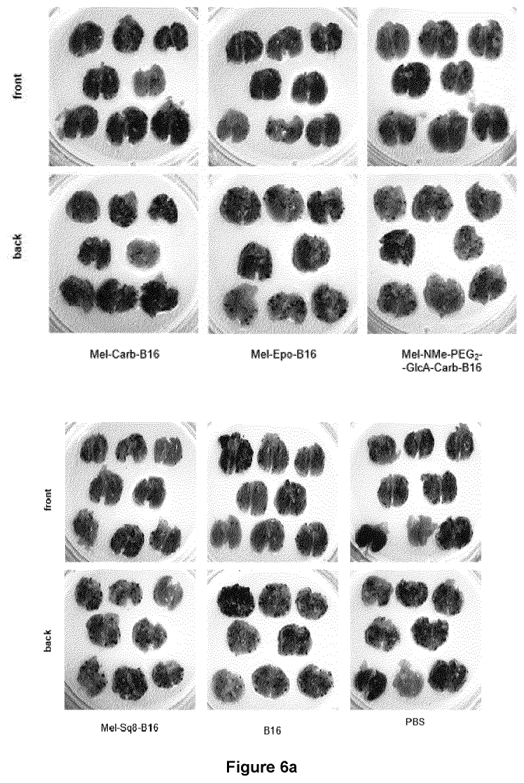

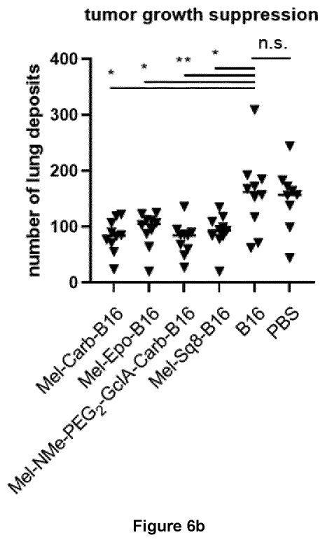

FIGS. 6a and 6b visualize the degree of lung metastasis in different test groups. Female C57BL/6 mice (Envigo) were subcutaneously immunized with 250 μg melibiose-ligated (melibiose carbamate (Mel-Carb), melibiose epoxide (Mel-Epo), melibiose N-methyl-(PEG)₂-glycuronic-acid-carbamate (Mel-NMe-PEG₂-GlcA-Carb) or melibiose squarate (Mel-Sq8) Keyhole limpet hemocyanin (KLH), non-ligated KLH or DPBS. 4 weeks after primary immunization animals received melibiose-ligated (melibiose carbamate (Mel-Carb), melibiose epoxide (Mel-Epo), melibiose N-methyl-(PEG)₂-glycuronic-acid-carbamate (Mel-NMe-PEG₂-GlcA-Carb) or melibiose squarate (Mel-Sq8) B16 cell lysate vaccines, non-ligated B16 cell lysate vaccines or DPBS. For both primary and booster vaccinations MPL/Alum adjuvant was used. 5 weeks after primary vaccination, tumorigenic B116.F10 cells were detached with 2 mM EDTA from running cell cultures (at 37° C. with 5% C02) and washed with sterile Dulbecco's phosphate buffered saline (DPBS). Live tumor cells were stored on ice and 2×10⁵ cells per recipient were injected via the tail vein (within 30 minutes of preparing the cell suspension). 16 days after tumor challenge animals were euthanized, and lungs were collected. Frontal and back images and quantitative representation of FIG. 6a visualizes the degree of lung metastasis in the tested groups. Unpaired non-parametric Mann-Whitney t-test was used to calculate statistical differences compared to the non-glycosylated vaccine-treated (B116) animals. n.s., not significant, P value>0.05; *P<0.05; **P<0.01 (see FIG. 6b).

1-13. (canceled)

14. A method of treatment of a subject suffering from cancer, the method comprising:
a. producing a glycoengineered tumor cell or tumor cell fragment comprising the steps:
i. obtaining a tumor cell or tumor cell fragment from the subject;
ii. in vitro glycoengineering the obtained tumor cell or tumor cell fragment by modifying a surface of the obtained tumor cell or tumor cell fragment with one or more carbohydrate antigen moieties to produce the glycoengineered tumor cell or tumor cell fragment;
b. administering the glycoengineered tumor cell or glycoengineered tumor cell fragment to the subject.

15. The method according to claim 14, wherein step a. is preceded by the steps: collecting a sample from the subject, the sample comprising anti-carbohydrate antibodies of the subject; in vitro determining and selecting endogenous anti-carbohydrate antibodies of the subject and identifying one or more carbohydrate antigen moieties corresponding to the selected endogenous anti-carbohydrate antibodies, wherein the identified one or more carbohydrates antigen moieties are in step a.ii. provided to the tumor cell surface of the obtained tumor cell or tumor cell fragment as the one or more carbohydrate antigen moieties.

16. The method according to claim 15, wherein the sample collected from the subject is a blood sample.

17. The method according to claim 15, wherein the identified one or more carbohydrate antigen moieties have an equilibrium constant K_{d}=k_{off}/kₒₙ with the endogenous anti-carbohydrate antibodies of less than 1 mM.

18. The method according to claim 15, wherein the identified one or more carbohydrate antigen moieties have an equilibrium constant K_{d}=k_{off}/kₒₙ with the endogenous anti-carbohydrate antibodies of less than 1 μM.

19. The method according to claim 14, wherein a tumor cell is obtained from the subject and is in vitro glycoengineered to produce a glycoengineered tumor cell, and step a. further comprises the step:
iii. disaggregating the glycoengineered tumor cell to produce the glycoengineered tumor cell fragment.

20. The method according to claim 14, wherein a tumor cell is obtained from the subject and is in vitro glycoengineered to produce a glycoengineered tumor cell, and the tumor cell is inactivated before, during or after invitro glycoengineering the obtained tumor cell such that it cannot divide further.

21. The method according to claim 14, wherein in-vitro glycoengineering the obtained tumor cell or tumor cell comprises a chemical ligation, enzymatic glycosylation or glycolipid cell membrane insertion.

22. The method according to claim 14, wherein the in vitro glycoengineering the obtained tumor cell or tumor cell comprises binding the one or more carbohydrate antigen moieties to the tumor cell surface via a urea group or via a 1,2-aminohydroxyethyl group.

23. The method according to claim 14, wherein the one or more carbohydrate antigen moieties are glycan moieties.

24. The method according to claim 23, wherein the glycan moieties are selected from Rha-, ManNAc-, GlcNAc-, Galβ1-4Glc-, Galβ1-6Galβ1-4Glc-, GlcNAcβ1-2Galβ1-3GalNAc-, GlcNAcβ1-6(GlcNAcβ1-4)GalNAc-, Galα1-6Glc-, Galα1-3GlcNAc-, GalNAcα1-3GalNAc-, GlcNAcβ1-4(6-O-Su)GlcNAc-, Galβ1-3GlcNAcα1-6Galβ1-4GlcNAc-, Galβ1-3GlcNAcα1-3Galβ1-3GlcNAc-, Galα1-4GlcNAc-, 6-Bn-Galα1-4(6-Bn)GlcNAc-, GlcNAcβ1-4-MurNAc-, GlcNAcβ1-4Mur-, GalNAcα1-3GalNAcβ1-3Gal-, Galα1-4GlcNAcβ1-3Galβ1-4GlcNAc-, Galβ1-3GlcN(Fm)β1-3Galβ1-4GlcNAc-, GlcNAcβ1-4GlcNAc-, Galα1-2Gal-, Galα1-3Galβ1-4Glc-, GlcNAcα1-3GalNAc-, Neu5Acα2-8Neu5Ac-, Fucα1-2Galβ1-4Glc-, Neu5Acα2-3Galβ1-4Glc-, GlcNAcβ1-4Galβ1-4GlcNAc-, Neu5Acα2-8Neu5Acα2-4Neu5Ac-, Galβ1-3GlcNAcβ1-3Galβ1-4Glc-, Galβ1-4GlcNAcβ1-3Galβ1-4Glc-, Galα1-3Galβ1-4GlcNAcβ1-3Galβ1-4Glc-, Arafβ1-2Arafα1-5(Arafβ1-2Arafα1-3)Arafα1-5Araf-, Neu5Acα2-8Neu5Acα2-4Neu5Acα2-6GlcNAcβ1-6GlcNAc-, Fucα1-4GlcNAc-, GalNAcα1-3GalNAcβ1-3Galα1-4Galβ1-4Glc-, GlcNAcα1-3Galβ1-4GlcNAc-, 3-O-Su-GlcNAc-, 6-O-Su-Gal-, GalNAcα1-3Gal-, GlcNAcβ1-3GalNAc-, (6-O-Bn-Galβ1)-3GlcNAc-, Galα1-3GalNAc(fur)-, 4,6-O-Su2-Galβ1-4GlcNAc-, GlcNAcβ1-6(GlcNAcβ1-3)GalNAc-, Galβ1-4Galβ1-4GlcNAc-, GalNAcα1-3Galβ1-4GlcNAc-, Galβ1-3GlcNAcα1-3Galβ1-4GlcNAc-, Galβ1-3GlcN(Fm)β1-3Galβ1-3GlcNAc-, Galβ1-3GalNAcβ1-3Galα1-4Galβ1-4Glc-, Galβ1-3GalNAc(fur)-, Fucα1-3GlcNAc-, Fucβ1-3GlcNAc-, Galβ1-3GlcNAc-, GlcNAcβ1-6GalNAc-, 3-O-Su-Galβ1-3GlcNAc-, 6-O-Su-Galβ1-3GlcNAc-, GlcAβ1-3GlcNAc-, 3,4-O-Su2-GalNAcβ1-4GlcNAc-, Galβ1-4GlcNAcβ1-6GalNAc-, and GlcNAcβ1-4Galβ1-4GlcNAc-.

25. The method according to claim 23, wherein the glycan moieties are not Gal, α-Gal or N-propionylated polysialic acid-polysialic glycans.

26. The method according to claim 14, wherein the tumor cell or tumor cell fragment is obtained from the subject by biopsy.

27. The method according to claim 14, wherein the tumor cell or tumor cell fragment is obtained by surgery.

28. The method according to claim 14, wherein the glycoengineered tumor cell fragment administered in step b. is a lysate obtained from lysing the glycoengineered tumor cell.
